SEQUENCE LISTING (62 sequences)

SEQ ID NO: 1 - DNA, 22 bp, Picornavirus
  misc_feature (1)..(22)
ctagtttgggtgtccgtgtttc

SEQ ID NO: 2 - DNA, 39 bp, artificial
  DNAzyme
  misc_feature (1)..(39)
gtgaaacacggacaggctagctacaacgaccaaagtagt

SEQ ID NO: 3 - DNA, 38 bp, artificial
  DNAzyme
  misc_feature (1)..(38)
tgaaacacggacaggctagctacaacgaccaaagtagt

SEQ ID NO: 4 - DNA, 37 bp, artificial
  DNAzyme
  misc_feature (1)..(37)
gaaacacggacaggctagctacaacgaccaaagtagt

SEQ ID NO: 5 - DNA, 36 bp, artificial
  DNAzyme
  misc_feature (1)..(36)
aaacacggacaggctagctacaacgaccaaagtagt

SEQ ID NO: 6 - DNA, 35 bp, artificial
  DNAzyme
  misc_feature (1)..(35)
aacacggacaggctagctacaacgaccaaagtagt

SEQ ID NO: 7 - DNA, 34 bp, artificial
  DNAzyme
  misc_feature (1)..(34)
acacggacaggctagctacaacgaccaaagtagt

SEQ ID NO: 8 - DNA, 33 bp, artificial
  DNAzyme
  misc_feature (1)..(33)
cacggacaggctagctacaacgaccaaagtagt

SEQ ID NO: 9 - DNA, 32 bp, artificial
  DNAzyme
  misc_feature (1)..(32)
acggacaggctagctacaacgaccaaagtagt

SEQ ID NO: 10 - DNA, 31 bp, artificial
  DNAzyme
  misc_feature (1)..(31)
cggacaggctagctacaacgaccaaagtagt

SEQ ID NO: 11 - DNA, 30 bp, artificial
  DNAzyme
  misc_feature (1)..(30)
ggacaggctagctacaacgaccaaagtagt

SEQ ID NO: 12 - DNA, 29 bp, artificial
  DNAzyme
  misc_feature (1)..(29)
gacaggctagctacaacgaccaaagtagt

SEQ ID NO: 13 - DNA, 28 bp, artificial
  DNAzyme
  misc_feature (1)..(28)
acaggctagctacaacgaccaaagtagt

SEQ ID NO: 14 - DNA, 27 bp, artificial
  DNAzyme
  misc_feature (1)..(27)
caggctagctacaacgaccaaagtagt

SEQ ID NO: 15 - DNA, 36 bp, artificial
  DNAzyme
  misc_feature (1)..(36)
gaaacacggacaggctagctacaacgaccaaagtag

SEQ ID NO: 16 - DNA, 35 bp, artificial
  DNAzyme
  misc_feature (1)..(35)
gaaacacggacaggctagctacaacgaccaaagta

SEQ ID NO: 17 - DNA, 34 bp, artificial
  DNAzyme
  misc_feature (1)..(34)
gaaacacggacaggctagctacaacgaccaaagt

SEQ ID NO: 18 - DNA, 33 bp, artificial
  DNAzyme
  misc_feature (1)..(33)
gaaacacggacaggctagctacaacgaccaaag

SEQ ID NO: 19 - DNA, 32 bp, artificial
  DNAzyme
  misc_feature (1)..(32)
gaaacacggacaggctagctacaacgaccaaa

SEQ ID NO: 20 - DNA, 31 bp, artificial
  DNAzyme
  misc_feature (1)..(31)
gaaacacggacaggctagctacaacgaccaa

SEQ ID NO: 21 - DNA, 30 bp, artificial
  DNAzyme
  misc_feature (1)..(30)
gaaacacggacaggctagctacaacgacca

SEQ ID NO: 22 - DNA, 29 bp, artificial
  DNAzyme
  misc_feature (1)..(29)
gaaacacggacaggctagctacaacgacc

SEQ ID NO: 23 - DNA, 38 bp, artificial
  DNAzyme
  misc_feature (1)..(38)
gtgaaacacggaggctagctacaacgaacccaaagtag

SEQ ID NO: 24 - DNA, 37 bp, artificial
  DNAzyme
  misc_feature (1)..(37)
tgaaacacggaggctagctacaacgaacccaaagtag

SEQ ID NO: 25 - DNA, 36 bp, artificial
  DNAzyme
  misc_feature (1)..(36)
gaaacacggaggctagctacaacgaacccaaagtag

SEQ ID NO: 26 - DNA, 35 bp, artificial
  DNAzyme
  misc_feature (1)..(35)
aaacacggaggctagctacaacgaacccaaagtag

SEQ ID NO: 27 - DNA, 34 bp, artificial
  DNAzyme
  misc_feature (1)..(34)
aacacggaggctagctacaacgaacccaaagtag

SEQ ID NO: 28 - DNA, 33 bp, artificial
  DNAzyme
  misc_feature (1)..(33)
acacggaggctagctacaacgaacccaaagtag

SEQ ID NO: 29 - DNA, 32 bp, artificial
  DNAzyme
  misc_feature (1)..(32)
cacggaggctagctacaacgaacccaaagtag

SEQ ID NO: 30 - DNA, 31 bp, artificial
  DNAzyme
  misc_feature (1)..(31)
acggaggctagctacaacgaacccaaagtag

SEQ ID NO: 31 - DNA, 30 bp, artificial
  DNAzyme
  misc_feature (1)..(30)
cggaggctagctacaacgaacccaaagtag

SEQ ID NO: 32 - DNA, 29 bp, artificial
  DNAzyme
  misc_feature (1)..(29)
ggaggctagctacaacgaacccaaagtag

SEQ ID NO: 33 - DNA, 28 bp, artificial
  DNAzyme
  misc_feature (1)..(28)
gaggctagctacaacgaacccaaagtag

SEQ ID NO: 34 - DNA, 37 bp, artificial
  DNAzyme
  misc_feature (1)..(37)
gaaacacggaggctagctacaacgaacccaaagtagt

SEQ ID NO: 35 - DNA, 35 bp, artificial
  DNAzyme
  misc_feature (1)..(35)
gaaacacggaggctagctacaacgaacccaaagta

SEQ ID NO: 36 - DNA, 34 bp, artificial
  DNAzyme
  misc_feature (1)..(34)
gaaacacggaggctagctacaacgaacccaaagt

SEQ ID NO: 37 - DNA, 33 bp, artificial
  DNAzyme
  misc_feature (1)..(33)
gaaacacggaggctagctacaacgaacccaaag

SEQ ID NO: 38 - DNA, 32 bp, artificial
  DNAzyme
  misc_feature (1)..(32)
gaaacacggaggctagctacaacgaacccaaa

SEQ ID NO: 39 - DNA, 31 bp, artificial
  DNAzyme
  misc_feature (1)..(31)
gaaacacggaggctagctacaacgaacccaa

SEQ ID NO: 40 - DNA, 30 bp, artificial
  DNAzyme
  misc_feature (1)..(30)
gaaacacggaggctagctacaacgaaccca

SEQ ID NO: 41 - DNA, 29 bp, artificial
  DNAzyme
  misc_feature (1)..(29)
gaaacacggaggctagctacaacgaaccc

SEQ ID NO: 42 - DNA, 28 bp, artificial
  DNAzyme
  misc_feature (1)..(28)
gaaacacggaggctagctacaacgaacc

SEQ ID NO: 43 - DNA, 27 bp, artificial
  DNAzyme
  misc_feature (1)..(27)
gaaacacggaggctagctacaacgaac

SEQ ID NO: 44 - DNA, 33 bp, artificial
  DNAzyme
  misc_feature (1)..(33)
gaaacaggctagctacaacgaggacacccaaag

SEQ ID NO: 45 - DNA, 32 bp, artificial
  DNAzyme
  misc_feature (1)..(32)
aaacaggctagctacaacgaggacacccaaag

SEQ ID NO: 46 - DNA, 31 bp, artificial
  DNAzyme
  misc_feature (1)..(31)
aacaggctagctacaacgaggacacccaaag

SEQ ID NO: 47 - DNA, 30 bp, artificial
  DNAzyme
  misc_feature (1)..(30)
acaggctagctacaacgaggacacccaaag

SEQ ID NO: 48 - DNA, 29 bp, artificial
  DNAzyme
  misc_feature (1)..(29)
caggctagctacaacgaggacacccaaag

SEQ ID NO: 49 - DNA, 34 bp, artificial
  DNAzyme
  misc_feature (1)..(34)
gaaacaggctagctacaacgaggacacccaaagt

SEQ ID NO: 50 - DNA, 32 bp, artificial
  DNAzyme
  misc_feature (1)..(32)
gaaacaggctagctacaacgaggacacccaaa

SEQ ID NO: 51 - DNA, 31 bp, artificial
  DNAzyme
  misc_feature (1)..(31)
gaaacaggctagctacaacgaggacacccaa

SEQ ID NO: 52 - DNA, 30 bp, artificial
  DNAzyme
  misc_feature (1)..(30)
gaaacaggctagctacaacgaggacaccca

SEQ ID NO: 53 - DNA, 29 bp, artificial
  DNAzyme
  misc_feature (1)..(29)
gaaacaggctagctacaacgaggacaccc

SEQ ID NO: 54 - DNA, 28 bp, artificial
  DNAzyme
  misc_feature (1)..(28)
gaaacaggctagctacaacgaggacacc

SEQ ID NO: 55 - DNA, 27 bp, artificial
  DNAzyme
  misc_feature (1)..(27)
gaaacaggctagctacaacgaggacac

SEQ ID NO: 56 - DNA, 26 bp, artificial
  DNAzyme
  misc_feature (1)..(27)
gaaacaggctagctacaacgaggaca

SEQ ID NO: 57 - DNA, 25 bp, artificial
  DNAzyme
  misc_feature (1)..(26)
gaaacaggctagctacaacgaggac

SEQ ID NO: 58 - DNA, 24 bp, artificial
  DNAzyme
  misc_feature (1)..(24)
gaaacaggctagctacaacgagga

SEQ ID NO: 59 - DNA, 23 bp, artificial
  DNAzyme
  misc_feature (1)..(23)
gaaacaggctagctacaacgagg

SEQ ID NO: 60 - DNA, 33 bp, artificial
  DNAzyme
  misc_feature (1)..(33)
acacggacaggctagctacaacgaccaaagtag

SEQ ID NO: 61 - DNA, 33 bp, artificial
  DNAzyme
  misc_feature (1)..(33)
aaacacggaggctagctacaacgaacccaaagt

SEQ ID NO: 62 - DNA, 33 bp, artificial
  DNAzyme
  misc_feature (1)..(33)
agtgaaacaggctagctacaacgaggacaccca

(19) United States
(12) Patent Application Publication
(10) Pub. No.: US 2018/0171339 A1
(43) Pub. Date: Jun. 21, 2018

(54) AGENT FOR THE PROPHYLAXIS AND THERAPY OF VIRAL INFECTION

(71) Applicant: SECARNA PHARMACEUTICALS GMBH & CO. KG, Marburg (DE)

(72) Inventors: Daniel POTACZEK, Marburg (DE); Holger GARN, Marburg (DE); Harald RENZ, Marburg (DE)

(73) Assignee: Secarna Pharmaceuticals GmbH & Co KG, Marburg (DE)

(21) Appl. No.: 15/574,944

(22) PCT Filed: May 13, 2016
(86) PCT No.: PCT/EP2016/060872

§ 371 Date: Feb. 13, 2018

(30) Foreign Application Priority Data
May 20, 2015 (EP) 15186512.0

(51) Int. Cl. C12N 15/113; A61P 31/14; A61P 31/16; A61K 31/711; A61K 45/06

(52) U.S. Cl. CPC C12N 15/1131; A61P 31/14; A61P 31/16; C12N 2320/31; A61K 45/06; C12N 2310/127; A61K 31/711

(57) ABSTRACT

The present invention relates to the preparation of a medicinal agent comprising DNAzymes according to Seq. ID 2-62 for the prophylaxis and treatment of virus infections that are caused by picornaviruses, in particular by rhinoviruses, in particular for the prophylaxis and treatment of rhinitis, head colds, asthma, COPD and viral infections of the upper respiratory tract.

The present invention relates to an agent for the prophylaxis and treatment of virus infections that are caused or triggered by picornaviruses, in particular by rhinoviruses.

### Picornaviruses

Picornaviruses are a special group of RNA viruses that trigger infections and diseases in humans and various other mammals. The viral genome of the picornaviruses includes a single stranded RNA with positive polarity. A single open reading frame for a viral precursor polyprotein is located between two non-coding regions at the 3′ end and 5′ end, which during the translation is cleaved into individual virus proteins. The poly-A tail typical of positive-strand viruses is located at the 3′ end. A region in which the RNA in has a complex secondary structure through numerous complementary base pairs is located at the 5′ end before the start codon. Functionally this section corresponds to an IRES (internal ribosomal entry site) for the initiation of the translation at the ribosomes.

The picornaviruses include:
Coxsackieviruses
Echoviruses
Enteroviruses
Polioviruses
Rhinoviruses

Infections with these viruses produce a number of clinically related diseases, such as for example aseptic meningitis, herpangina (also Zahorsky disease), hand foot and mouth disease, haemorrhagic conjunctivitis, diseases of the respiratory tract (e.g. summer flue, pharyngitis, pneumonia) and diseases of the internal organs (e.g. pericarditis, myocarditis, pleurodynia).

### Diagnosis

On account of the large number of possible causative agents in question, a reliable diagnosis in a biological sample obtained from a human or mammal is still carried out by direct detection of the causative agent by means of virus cultivation in cell culture followed by type identification (neutralisations test) and detection of the virus genome with molecular methods (nucleic acid amplification techniques, such as e.g. RT-PCR). This direct detection in a biological sample such as blood, stools, fluid or a pharyngeal wash is very complicated and expensive.

### Treatment

Also, a specific drug treatment of virus infections is not possible at the present time. Treatment is carried out only symptomatically and is directed at the affected organ system.

### Prophylaxis

There are a number of prophylactic hygiene measures for preventing a virus infection. The most important protection against infections and diseases is vaccination. Such a vaccination protection against polio viruses is available, though no vaccines or only relatively ineffective vaccines are available for rhinoviruses and other viruses of the group of Picornaviridae.

### Rhinoviruses

The group of rhinoviruses include ca. 157 different types, which are subdivided into three groups A, B and C depending on the homology in the genome. Rhinoviruses of groups A and B bind mostly to the cellular ICAM-1 receptor or to the LDL receptor.

The viral proteins are produced as polyprotein and are then cleaved by proteases. The structural proteins located in the virion are coded in the 5′ region of the RNA, the non-structural proteins in the 3′ region. At the 5′ end there is a non-translated region (5′-UTR) to which the P1 region, the P2 region and the P3 region are joined, which respectively code for capsid proteins (VP1 to VP4), protease and RNA polymerase. This is followed by a further non-translated region (3′-UTR) and a poly-A tail. The capsid proteins (VP1 to VP4) serve for the packaging of the genome and also as a receptor for the attachment to a host cell. VP1-3 are recognised as surface proteins of antibodies of the host organism.

Rhinoviruses are widespread throughout the world and are restricted to humans. They prefer temperatures of 3° C. to 33° C. for their reproduction, but also reproduce at higher temperatures, especially if the infections affect not only the mucus membrane region but also the respiratory pathway and pulmonary region.

Rhinitis acuta (common cold) is the most common infectious disease caused by rhinoviruses in humans, and results in colds, asthma or an increase in respiratory tract sensitivity. Viral infections of the upper respiratory tract are involved in up to 40% of all acute asthma attacks or exacerbated COPD (chronic obstructive pulmonary disease). The viruses damage the epithelial layer of the respiratory tract, trigger an inflammation and increase the neurosensitivity of the respiratory tract cells. In asthmatics this leads to serious consequences, but also healthy people are affected. As a result many people suffer from a persistent cough following a “cold”.

Whereas infections with rhinoviruses in healthy people usually produce a harmless cold that quickly abates, infections in asthmatics and people who already suffer from a sensitivity of the respiratory tract cells can be very severe and can trigger life-threatening dyspnoea, stenocardia and shortness of breath.

British scientists recently showed that infections with rhinoviruses trigger an increased formation of cytokines, in particular interleukin (IL-25), in pulmonary epithelial cells of humans. This leads to a signal cascade as in the case of an allergic reaction and ultimately to an unusually severe inflammatory reaction.

In this connection it is also known that infection with rhinoviruses in children leads to a sensitivity of the respiratory tract cells and to a higher incidence of asthma in adulthood.

### Prophylaxis

The prevailing scientific opinion is that prophylaxis before an infection with rhinoviruses, as well as causal treatment, are hardly possible, which means that it is left the person's immune system to deal with the infection. However, the immune system of neonates in particular, and patients with natural or drug-related immune suppression, is extremely weakened, so that a prophylaxis before an infection with picornaviruses, in particular rhinoviruses, is desirable.

### Treatment

There is no causal treatment, which inevitably means that an affected person has to suffer the course of the illness. Only agents for a symptomatic treatment of the accompanying symptoms of a head cold and headache are available. Known symptomatic measures include physical rest, inhalations, rubbing with ethereal oils and healthy eating. There are also various nasal pharmaceutical formulations containing active constituents that are supposed to act as a decongestant or have a soothing effect on the nasal mucosa. The person skilled in the art knows of nasal sprays containing the active constituents tramazoline and xylometazoline, which free the respiratory tract for a short time. Nasal sprays containing the active constituent oxymetazoline have a direct antiviral effect by preventing the expression of ICAM-1, the receptor for rhinoviruses. However, these are active only against head colds and are not effective in the case of severe viral infections such as asthma or COPD (chronic obstructive pulmonary disease). Furthermore lozenges to treat sore throats and effervescent tablets, ointments or drops containing active constituents that are intended to facilitate expectoration of thick mucus or ease coughing are used, and in some cases analgesics and antipyretics are also recommended for a short period to the patient. In many cases a viral infection is also followed by a bacterial superinfection and therefore also by pulmonary inflammation. These are then treated with various antibiotics, in which the effects of antibiotic resistance are generally known. In the treatment of asthma or COPD replacing oral treatment by systemic treatment may even be necessary, in which case glucocorticoids have to be used and hospital treatment is expensive and complicated.

### Vaccination

Up to now there is no specific vaccination against rhinoviruses. In the 1960s various vaccines were produced, which however only provided a specific vaccination protection against the particular seasonal or regional rhinovirus or viruses.

However, also a rhinovirus infection in the body produces only an immunity against the specific type of infection. A characteristic property of RNA viruses in general is their increased mutation rate and the resultant flexibility. Thus, for example, the capsid proteins of rhinoviruses are extremely variable at the protein level, which seriously complicates the formation of a general immunity, and furthermore up to now it has not been possible to produce a vaccine that is effective against more than one type of rhinovirus. Presently there is no specific medication or vaccines that are effective over the long term. Although the active constituent pleconaril, a capsid blocker that interacts with the capsid protein VP1, is mentioned in the prior art, a commercial preparation is however not available or authorised.

### DNAzymes

DNAzymes are catalytically active single stranded DNA molecules.

A known DNAzyme family are the “10-23” DNAzymes, which specifically recognise the target sequences of RNA molecules, bind in a complementary fashion and cleave by catalytic activity, in which the activity of the cleaved RNA molecule is lost or reduced. Therapeutically this is a highly promising approach for the treatment of diseases in humans or animals that are caused by enhanced expression of RNA molecules. A precondition is however that the target structure or the target sequence for the binding is sufficiently identified and accessible and that the DNAzymes can manifest a good binding property and also catalytic activity.

10-23-DNAzymes have a catalytic domain of 15 nucleotides, which is flanked by two substrate binding domains (I and II). The binding to the RNA substrate takes place by base pairing according to the Watson-Crick rules via the substrate binding domains I and II.

### PRIOR ART

It is known to use antisense strategy in order to bind and block pathogenic RNA molecules in the human or animal body.

The prior art knows “10-23” DNAzymes, which recognise, bind and cut the highly specific RNA target structures. The target structure is thereby inactivated, inhibited and blocked, so that its pathogenic function can no longer be exerted in the organism. Selected DNAzymes are described for medicinal use as agents for inhibiting virus replication for example. These DNAzymes are however directed very specifically against conserved regions of specific RNA viruses of one type and are therefore not able to cover a group of multiple types of viruses or highly variable types of viruses. They have no broad applicability in multiple types of viruses or in highly variable types of viruses, such as the picornaviruses.

DE103 22 662A1 also discloses DNAzymes modified by the “10-23” DNAzyme. These recognise and bind to a target sequence IRES of rhinovirus of the serotype HRV14.

The disadvantage is that this target sequence is less conserved and is subject to a high mutation rate. Accordingly the effectiveness of the DNAzymes is restricted only to rhinoviruses of the serotype HRV14 and they are not generally effective for all serotypes of rhinoviruses or all serotypes of picornaviruses.

In US 20110091501 improved rhinovirus vectors and their sequences are described, which are employed and used as transport vehicles for immunogens, e.g. influenza virus immunogens, in treatment. The disclosed vector is disclosed with the nucleotide sequences of the rhinovirus of the serotype HRV14. It is not used however as an antisense molecule in order to bind rhinovirus mRNA.

US 20130309238 discloses one possibility of realising the rhinovirus-associated inflammatory reactions and asthma, by antisense blockade of the midline-1 reaction path. Although a catalytic antisense construct is mentioned here, there is no mention of specific DNAzymes against rhinoviruses of special serotypes or as many serotypes as possible.

None of the discovered citations from the prior art discloses specific DNAzymes that can be used against a large number of virus infections that are triggered by picornaviruses, in particular rhinoviruses.

### Object

The object of the present invention is to eliminate the disadvantages in the prior art and provide an agent for the prophylaxis and treatment of virus infections that are triggered by picornaviruses, in particular rhinoviruses, and that is effective against a large number of serotypes.

### Achievement of the Object

This object is achieved according to the invention by at least one DNAzyme according to SeqID 2-62 and its use according to the invention in accordance with the features of claim 1 and the dependent claims.

### 1. Target Sequence for Binding the DNAzymes According to the Invention

Surprisingly, in an alignment of all available genome sequences of picornaviruses evaluated by means of bioinformatics a very high homology and sequence identity was identified in the 5′-UTR region of the virus genome. This region is highly conserved and exhibits a low mutation rate within the viruses.

More accurate evaluations of all available genome sequences of rhinoviruses (according to McIntyre et al. J Gen Virol 2012) also show a very high homology and sequence identity in this 5′-UTR region.

This homologous 5′-UTR region Seq. ID 1 thus represents a good starting point for providing DNAzymes that are complementary to and specific for mRNA of as many picornaviruses as possible and as many serotypes of rhinoviruses as possible.

The DNAzymes according to the invention are directed against this mRNA from the 5′-UTR region of the virus genome. A cleavage of this mRNA by at least one of the DNAzymes according to the invention inhibits the replication of many picornaviruses and many serotypes of rhinoviruses and thus acts against the outbreak of the disease.

The DNAzymes according to the invention are directed specifically against this target sequence. They therefore form an effective agent for the prophylaxis and treatment of virus infections that are triggered by picornaviruses, in particular rhinoviruses. They are effective against a large number of serotypes.

In particular the DNAzymes according to the invention that are selected from the group dpp-X1, dpp-X2 and dpp-j-9 (Seq. ID 2-62) have unexpectedly positive properties with regard to
binding to the target sequence in the 5′-UTR region of the virus genome
high enzymatic cleavage activity on the target sequence in the 5′-UTR region
high inactivation of the virus RNA in various serotypes of rhinoviruses and other picornaviruses

The cleavage of the DNAzymes according to the invention is determined on all available genome sequences of picornaviruses, in particular rhinoviruses (according to Mcintyre et al. J Gen Virol 2012).

A 97.4% homology and thus cleavage is found in all HRV-A strains. A 100% homology and thus cleavage is found all HRV-B strains. A 98.08% homology and thus cleavage is found in all HRV-C strains.

### 2. DNAzymes

### Structure of the DNAzymes According to the Invention

The DNAzymes selected from the group dpp-X1, dpp-X2 and dpp-j-9 (Seq. ID 2-62) comprise in each case a central catalytic domain of 15 deoxyribonucleic acids with the sequence GGCTAGCTACAACGA, which is flanked by two substrate binding domains I and II. The two substrate binding domains I and II are important for the catalytic activity of the DNAzyme in the cleavage of the target sequence by de-esterification. They recognise the specific target sequence in the 5′-UTR region of the virus genome and bind very specifically via Watson-Crick base pairing.

The DNAzymes according to the invention recognise conserved RNA regions of the genome of picornaviruses, in particular of rhinoviruses, bind these regions in a complementary manner and cut these regions. The RNA of picornaviruses, in particular of rhinoviruses, is thereby inactivated, inhibited or blocked, so that their replication and thus the pathogenic action can no longer be manifested in the organism. The DNAzymes according to the invention represent an effective agent for medical use in order to inhibit virus replication of picornaviruses, in particular of rhinoviruses. The medical use for the prophylaxis and treatment is conducted on humans and/or mammals.

In particular the DNAzymes according to the invention that are selected from the group dpp-X1, dpp-X2 and dpp-j-9 (Seq. ID 2-62) have unexpectedly positive properties with regard to
binding to the target sequence in the 5′-UTR region of the virus genome
high enzymatic cleavage activity on the target sequence in the 5′-UTR region
high inactivation of the virus RNA in various serotypes of rhinoviruses and other picornaviruses

The length and sequence of the substrate binding domains I and II is critical for the cleavage properties and for the catalytic activity of the DNAzymes. The substrate binding domains I and II are either of the same length or of different lengths. Numerous series of tests have been carried out in order to demonstrate for the DNAzymes according to the invention selected from the group dpp-X1, dpp-X2 and dpp-j-9 (Seq. ID 2-62) different lengths and sequences of the substrate binding domains I and II with regard to their cleavage properties on the target sequence. The results of these cleavage experiments are shown in the exemplary embodiments.

In one implementation the substrate binding domains I and II are completely complementary to the special target sequence in the 5′-UTR region of the virus genome. In order to cleave RNA in the 5′-UTR region of the virus genome, the substrate binding domains I and II of the DNAzymes according to the invention do not have to be completely complementary however. In vitro investigations show that the DNAzymes according to the invention with a substrate domain I or II that have a homology of 80%, 85%, 90% or 95%, also bind to the target sequence in the 5′-UTR region of the virus genome and are capable of cleaving these.

### Modifications

Furthermore it was surprisingly found that the DNAzymes according to the invention that are selected from the group dpp-X1, dpp-X2 and dpp-j-9 (Seq. ID 2-62) are stabilised by modifications against nucleolytic attacks. This is important in particular for their use as a medical agent.

The effect of the modification is that the stabilised DNAzymes do not exhibit any significant reduction of the catalytic activity or even have an improved catalytic activity with regard to the respective RNA substrate.

A modified nucleotide includes in particular a chemical modification. The person skilled in the art understands this to mean that the modified nucleotide is altered by the removal, addition or replacement of individual or several atoms or groups of atoms compared to naturally occurring nucleotides. The chemical modification includes for example the ribose (e.g. 2′-O-methyl-ribonucleotides, so called “Locked Nucleic Acids” (LNA) ribonucleotides and inverted thymidine), the phosphorus (di)ester bond (e.g. phosphorus thioates, phosphorus amidates, methyl phosphonates and peptide nucleotides) and/or the base (e.g. 7-deazaguanosine, 5-methylcytosine and inosine).

In a particularly preferred embodiment the DNAzymes according to the invention can be modified at least one nucleotide of the substrate binding domain I and/or II, in particular by phosphorus thioate, inverted thymidine, 2′-O-methyl-ribose or LNA ribonucleotides. In the case of furthermore preferred DNAzymes a nucleotide or several nucleotides of the catalytic domains are modified, in particular by phosphorus thioate, inverted thymidine, 2′-O-methyl ribose or LNA ribonucleotides.

A preferred implementation is the introduction of a 3′-3′-Inversion at one end of the DNAzymes according to the invention. The term 3′-3′ inversion denotes a covalent phosphate bond between the 3′ carbon atoms of the terminal nucleotide and of the adjoining nucleotide. This type of bond is in contrast to the normal phosphate bond between the 3′ and 5′ carbon atoms of successive nucleotides. Accordingly it is preferred if the nucleotide at the 3′ end is the inverse of the substrate binding domain adjoining the 3′ end of the catalytic domain. In addition to the inversions the DNAzymes can contain modified nucleotides or nucleotide compounds. Modified nucleotides include for example N3′-P5′-phosphorus amidate compounds, 2′-O-methyl substitutions and peptide nucleic acid compounds. The production of all modifications is known to the person skilled in the art in this field and instructions regarding the procedure can be found in the prior art.

In one embodiment the modification is localised in the catalytic domain. In a further embodiment the modification is localised in the substrate binding domain I and/or II. In yet a further embodiment the modification is localised in the catalytic domain and in the substrate domain I and/or II.

### Use as Medicament

At least one of the DNAzymes according to the invention that are selected from the group dpp-X1, dpp-X2 and dpp-j-9 (Seq. ID 2-62) with or without modifications is a component of a medicinal agent for the prophylaxis and treatment of virus infections that are caused or triggered by picornaviruses, in particular by rhinoviruses.

The agent according to the invention includes alternatively a pharmacologically compatible carrier, adjuvant and/or solvent.

The agent according to the invention is produced and administered in the form of drops, mouth spray, nasal spray, pills, tablets, film-coated tablets, layered tablets, suppositories, gels, ointments, syrup, inhalation powders, granules, emulsions, dispersions, microcapsules, capsules, powders or injection solutions. This also includes formulations such as layered tablets for the controlled and/or continuous release, as well as micro-encapsulations as special application form.

The agent according to the invention includes encapsulations in vesicles, as are known in dermatology and pharmacy for transport into the skin, for example: anionic or cationic liposomes, niosomes, nanoparticles or multilamellar vesicles for penetration through the skin or into the cells of the skin or hair.

The agent according to the invention is suitable inter alia for inhalation or for intravenous, intraperitoneal, intramuscular, subcutaneous, mucocutaneous, oral, rectal, transdermal, topical, buccal, intradermal, intragastral, intracutaneous, intranasal, intrabuccal, percutaneous or sublingual administration.

As pharmacologically compatible carriers there are used for example lactose, starch, sorbitol, sucrose, cellulose, magnesium stearate, dicalcium phosphate, calcium sulphate, talcum, mannitol, ethyl alcohol and the like. Powders as well as tablets can consist of 5% to 95% of such a carrier.

Furthermore disintegrants, colourants, flavouring agents and/or binders can be added to the agent according to the invention.

Liquid formulations include solutions, suspensions, sprays and emulsions, for example water-based or water/propylene glycol-based injection solutions for parenteral injections.

Capsules are produced for example from methyl cellulose, polyvinyl alcohols or denatured gelatines or starch.

As disintegrants there are used starch, sodium carboxymethyl starch, natural and synthetic gums, such as for example carob bean gum, karaya, guar bean, tragacanth and agar, as well as cellulose derivatives such as methylcellulose, sodium carboxymethylcellulose, microcrystalline cellulose and also alginates, clay earths and bentonites. These components are used in amounts of 2 to 30 wt. %.

Binders known to the person skilled in the art include sugars, starch obtained from corn, rice or potatoes, natural gums such as acacia gum, gelatins, tragacanths, alginic acid, sodium alginate, ammonium calcium alginate, methylcellulose, waxes, sodium carboxymethylcellulose, hydroxypropyl methylcellulose, polyethylene glycol, polyvinyl pyrrolidone as well as inorganic compounds such as magnesium aluminium silicates, which can be added to the agent according to the invention. The binders are normally added in amounts of 1 to 30 wt. %.

As lubricants there are known and used boric acid and stearates such as magnesium stearate, calcium stearate, potassium stearate, stearic acid, high melting point waxes as well as water-soluble lubricants such as sodium chloride, sodium benzoate, sodium acetate, sodium oleate, polyethylene glycol and amino acids such as leucine. Such lubricants are normally used in amounts of 0.05 to 15 wt. %.

The nature of the dosing of the agent according to the invention is determined by the treating physician depending on the clinical factors. It is known to the person skilled in the art that the type of dosing depends on various factors, such as for example body size, weight, body surface area, age, sex, or the general health of the patient, but also on the agent to be specifically administered, the duration and nature of the administration and on other medication that may possibly be administered in parallel.

The medicinal agent is suitable for the prophylaxis and treatment of virus infections that are caused by picornaviruses, especially by rhinoviruses, and in particular is also used for the prophylaxis and treatment of rhinitis, the common cold, asthma, COPD, and also for the prophylaxis and treatment of viral infections of the upper respiratory tract.

Surprisingly it was found that with at least one of the DNAzymes according to the invention that are selected from the group dpp-X1, dpp-X2 and dpp-j-9 (Seq. ID 2-62) a general prophylaxis is also achieved against the occurrence and manifestation of asthma and COPD.

A combination of the at least one DNAzyme according to the invention that is selected from the group dpp-X1, dpp-X2 and dpp-j-9 (Seq. ID 2-62) with at least one active constituent, for example analgesics and antipyretics, is possible. Furthermore the combination with at least one anti-inflammatory agent, immune modulator, antiasthmatic and/or bronchodilator, is possible.

Likewise, the combination with at least one antiparasitic, antibacterial, antimycotic and/or antiviral active constituent is possible.

Alternatively the combination with at least one dermatological or cosmetic active constituent is possible.

The agent is applied to a patient nasally, preferably in the form of solutions and/or emulsions. For this purpose it can be formulated as nasal drops or as a nasal spray. These nasally applicable agents act either to treat or to prevent infection in the nose itself or lead to the uptake of the agent in the bloodstream, so that it can exert its action at other sites in the body.

They alternatively contain adjuvants to stabilise the active constituent and/or to maintain a certain physiologically acceptable pH value in the nose. For this purpose the person skilled in the art knows that phosphate or phosphate/citrate or citrate buffers and also acetate buffers are suitable. Further adjuvants can be agents for oral and throat disinfection or preservatives.

### EXEMPLARY EMBODIMENTS

### 1. Alignment in Order to Identify a Best Possible Target Sequence in the 5′-UTR Region of the Virus Genome of Picornaviruses

The alignments of all available genome sequences of picornaviruses evaluated by means of bioinformatics show an extremely good homology and sequence identity in the 5′-UTR region of the virus genome.

The sequences are derived from sources that are known to the person skilled in the art, for example
Enterovirus A: http://www.picornaviridae.com/enterovirus/ev-a/ev-a.htm plus http://www.picornaviridae.com/enterovirus/ev-a/ev-a_segs.htm
Enterovirus B: http://www.picornaviridae.com/enterovirus/ev-b/ev-b.htm
Enterovirus C: http://www.picornaviridae.com/enterovirus/ev-c/ev-c.htm
Enterovirus D: http://www.picornaviridae.com/enterovirus/ev-d/ev-d.htm
Enterovirus E: http://www.picornaviridae.com/enterovirus/ev-e/ev-e.htm
Enterovirus F: http://www.picornaviridae.com/enterovirus/ev-f/ev-f.htm
Enterovirus G: http://www.picornaviridae.com/enterovirus/ev-g/ev-g.htm
Enterovirus H: http://www.picornaviridae.com/enterovirus/ev-h/ev-h.htm
Enterovirus J: http://www.picornaviridae.com/enterovirus/ev-j/ev-j.htm

This region is very well conserved within the picornaviruses and has a very low mutation rate. Accurate evaluations of all available genome sequences of rhinoviruses according to McIntyre et al. J Gen Virol 2012 also show a very good homology and sequence identity in this 5′-UTR region.

This homologous 5′-UTR region thus represents a good starting point for providing DNAzymes that bind at this target sequence to as many picornaviruses as possible and to as many serotypes of rhinoviruses as possible, and specifically cleave these.

The 5′-UTR region with the greatest homology is determined as target sequence for the binding of the DNAzymes according to the invention.

The GT (U) cleaving nucleotides are shown in bold type and are underlined:

Seq ID 1
CTAGTTTGGGTGTCCGTGTTTC

It was found that the DNAzymes according to the invention that are selected from the group dpp-X1, dpp-X2 and dpp-j-9 SeqID 2-62 bind specifically to the target sequence of human enteroviruses (A-D), but also to porcine enteroviruses (G) and simian enteroviruses (J) and cleave these.

### 2. Plasmid Construction

The genomic RNA of all available picornaviruses is isolated according to a method known to the person skilled in the art or with a commercially available kit, e.g. the QIAamp UltraSens Virus Kit, according to the manufacturers instructions, and is used for cDNA synthesis. The synthesis of DNA is carried out according to a method known to the person skilled in the art or using a commercially available kit, e.g. the Omniscript Reverse Transcription Kit (Qiagen) and addition of an RNaseOUT ribonuclease inhibitor (Invitrogen, Carlsbad, Calif., USA), according to the manufacturers instructions. For example, this is shown here on genomic RNA of rhinovirus serotype HRV-1b, -16 and -29.

The cDNA for each virus is used for the amplification of the specific homologous 5′-UTR region. For this, PCR reactions are carried out according to a method known to the person skilled in the art, or using a commercially available kit, for example with HotStarTaq Master Mix Kit (Qiagen) and sequence-specific primers. The PCR products that are obtained are separated by gel electrophoresis (2% agarose and 1×TBE TRIS-borate-EDTA buffer) and are extracted from the gel by a known method or using a commercially available kit, e.g. the Qiaquick Gel Extraction Kit (Qiagen), and stored at −20° C. The purified PCR products are subcloned according to conventional methods in a RNA expression vector, e.g. pGEM-T Easy Vector System II. The ligation product is transformed in competent cells, e.g. JM109 High Efficiency Competent cells. Culturing is then carried out in a suitable medium, e.g. SOC medium, and the transformed bacteria are plated out on agar plates, e.g. standard LB agar with ampicillin and cultured to a suitable density.

Positive cultures are transferred from the plates in mini-preps in standard LB medium with ampicillin and cultured. Plasmid DNA is isolated using a known method or with a commercially available kit, e.g. with the QIAprep Spin Miniprep Kit (Qiagen). The first verification of the cloning efficiency is carried out with EcoRI (FastDigest EcoRI, Therma Fisher Scientific), gel electrophoresis (1.5% agarose and 1×TBE TRIS-Borate-EDTA buffer), and sequencing with standard primer SP6. Bacteria that contain the desired sequence are cultured in maxi-preps (standard LB medium, ampicillin). The plasmid DNA is carried out by a known method or using a commercially obtainable kit, e.g. the HiSpeed Plasmid Maxi Kit (Qiagen). Purified plasmids are stored at −20° C. Alternatively a control sequencing is carried out with a standard SP6 primer.

### 3. RNA Expression (Synthesis)

The plasmids are linearised by known methods or with a commercially obtainable kit, using restriction enzymes, e.g. SpeI oder NcoI. The samples are precipitated with ethanol, EDTA and ammonium acetate and are tested for their linearisation efficiency in gel electrophoresis (0.8% agarose and 1×TBE TRIS-borat-EDTA buffer).

In vitro transcription is carried out by a known method or with a commercially obtainable kit, e.g. the Ambion MEGAscript T7 transcription kit or the Ambion MEGAscript SP6 transcription kit, according to the manufacturers instructions. RNA is purified by a known method or by using a commercially obtainable kit, e.g. the RNeasy Mini Kit (Qiagen). RNA samples are checked for their concentration by a known method or by using a commercially obtainable kit, e.g. NanoDrop 2000c (Thermo Fisher Scientific) and are analysed by gel electrophoresis (2.5% agarose, 1×TAE TRIS-Acetate-EDTA buffer). The thereby obtained RNA molecules correspond in their sequence to the insert surrounded by vector fragments between the T7 or SP6 polymerase transcription start sites and 5′ (genomic) end from the insert and between 3′ (genomic) end from the insert and SpeI or NcoI cleavage site.

In all experiments the binding of the DNAzymes according to the invention (Seq. ID 2-62) at the 5′-UTR region of the following types of picornaviruses is determined:

A1, A2, A7, A8, A9, A10, A11, A12, A13, A15, A16, A18, A19, A20, A21, A22, A23, A24, A25, A28, A29, A30, A31, A32, A33, A34, A36, A38, A39, A40, A41, A43, A45, A46, A47, A49, A50, A51, A53, A54, A55, A56, A57, A58, A59, A60, A61, A62, A63, A64, A65, A66, A67, A68, A71, A73, A74, A75, A76, A77, A78, A80, A81, A82, A88, A89, A90, A94, A96, A100, A101, A102, A103, A104, A105, A106,

B3, B4, B5, B6, B14, B17, B26, B27, B35, B37, B42, B48, B52, B69, B70, B72, B79, B83, B84, B86, B91, B92, B93, B97, B99, B100, B101, B102, B103, B104,

C1, C2, C3, C4, C5, C6, C7, C8, C9, C10, C11, C12, C15, C17, C22, C25, C26, C28, C32, C34, C35, C36, C38 C39, C40, C41, C42, C43, C45, C49, C51.

### 4. Preparation of the DNAzymes According to the Invention

The production of the DNAzymes according to the invention (Seq. ID 2-62) is carried out according to a synthesis method known to the person skilled in the art.

### 5. Cleavage Assays for Characterising the DNAzymes According to the Invention at the Target Sequence

The cleavage assays include a qualitative and quantitative analysis of the degree to which the DNAzymes according to the invention of the (Seq. ID 2-62) bind and cleave the target sequence in the 5′-UTR region of the virus genome of the picornaviruses, in particular of rhinoviruses.

The cleavage reaction is carried out with 1 μl reaction buffer, e.g. 500 mM TRIS, 1 μl 1 M NaCl, 1 μl 10 mM MgCl₂) and varying amounts of RNA and DNAzymes (between 50 and 250 ng or between 10 and 30 pmol) and double the amount of distilled water (up to 10 μl of the total volume). In quantitative experiments a reference RNA (e.g.: GATA3 mRNA) is added as control to the reaction mixture. The reaction mixtures are incubated at 37° C. for 60 min, then poured onto ice, and are denatured by adding Ambion Gel Loading Puffer II (Thermo Fisher Scientific) in order to stop the reaction.

After 10 minutes' incubation at 65° C. the reaction mixtures are separated by gel electrophoresis (2.5% agarose, 1×TAE buffer) and illustrated graphically, for example in the Fusion Fx7 System (PeqLab). For the quantitative determination the gel images are then also evaluated by band density measurement, e.g. with the Lablmage 1D L340 Bio-lmaging. The “rolling ball mode” is used to reduce the background.

### 6. Elongation and Shortening of the Substrate Binding Domains I and II

The inventors have in the course of their own research work produced a series of special DNAzymes with specific substrate binding domains I and II and have analysed their effectiveness on the special target sequence in the 5′-UTR region of the virus genome with various serotypes of rhinoviruses and other picornaviruses. The effectiveness is illustrated in binding and cleavage assays.

In particular the DNAzymes selected from the group dpp-X1, dpp-X2 and dpp-j-9 (Seq. ID 2-62) exhibit unexpectedly positive properties with respect to
binding to the target sequence in the 5′-UTR region of the virus genome
strong enzymatic cleavage activity on the target sequence in the 5′-UTR region
marked inactivation of the virus RNA with various serotypes of rhinoviruses and other picornaviruses

The efficiency of the cleavage of the DNAzymes according to the invention at the target sequence in the 5′-UTR region of the virus genome is illustrated in a cleavage assay with RNA from available picornaviruses, e.g. the rhinovirus types HRV-1b, -16 and -29.

| | | | Cleavage in serotypes | | |
|---|---|---|---|---|---|
| | | | of rhinoviruses in | | |
| DNAzyme | | | the 5′-UTR region | | |
| | Substrate | Substrate | HRV 1B | HRV 16 | HRV 29 |
| | binding | binding | RNA | RNA | RNA |
| Name | domain I | domain II | Fragment | Fragment | Fragment |
| SeqID | ACACGGACA | CCAAAGTAG | + | + | + |
| 60 | | | | | |
| dpp-X1 | | | | | |
| SeqID | AAACACGGA | ACCCAAAGT | + | + | + |
| 61 | | | | | |
| dpp-X2 | | | | | |
| SeqID | AGTGAAACA | GGACACCCA | ++ | + | + |
| 62 | | | | | |
| dpp- | | | | | |
| j-9 | | | | | |

The catalytic domain comprises a sequence of GGCTAGCTACAACGA.

It was also found that the sequence succession GT of the DNAzymes according to the invention is very important, since modification with a substitution at GC no longer showed binding to the target sequence in the 5′-UTR region of the virus genome. In the DNAzymes according to the invention selected from the group dpp-X1, dpp-X2 and dpp-j-9 (Seq. ID 2-62) length variations were carried out in the substrate binding domains I and II, the catalytic domain remaining unchanged.

### a) DNAzyme Dpp-X1 Shows a Good RNA Cleavage Activity of 97.76% of the Available Picornaviruses, e.g. the Rhinovirus Types HRV-1b, -16 and -29.

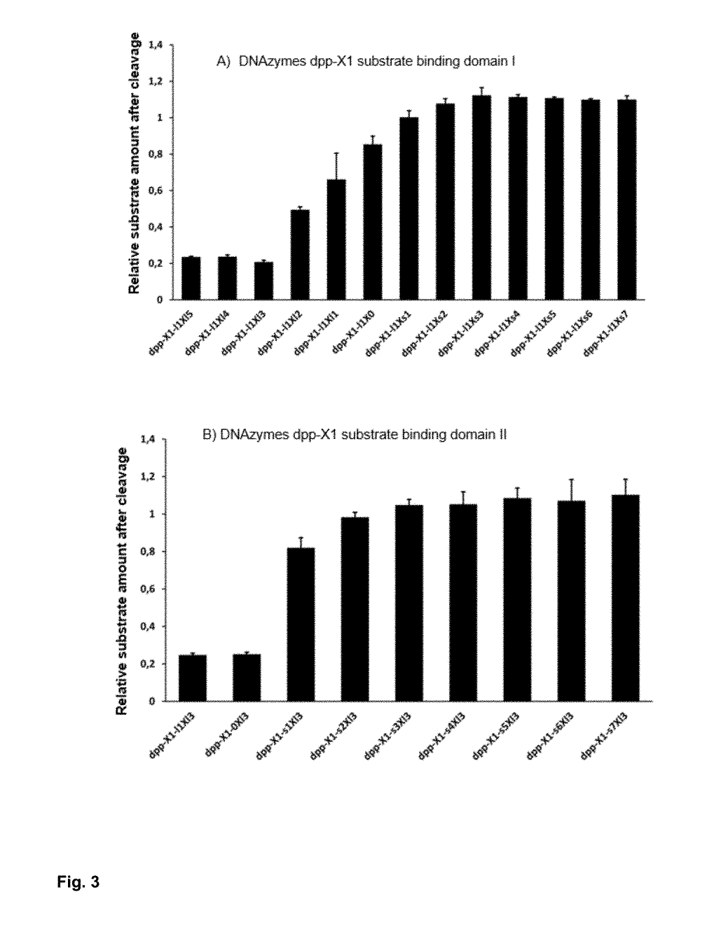

The following length variants of the substrate binding domain I were carried out, with a constant 10 nucleotides in the substrate binding domain II and the catalytic domain. The cleavage properties are tested in the cleavage assay, FIG. 3 A:

DNAzyme

| | | Substrate | | Substrate |
|---|---|---|---|---|
| Seq | | binding- | Catalytic | binding |
| ID | Name | domain I | domain | domain II |
| 2 | dpp-X1-l1X15 | GTGAAACACGGACA | GGCTAGC | CCAAAGTAGT |
| | | | TACAACGA | |
| 3 | dpp-X1-l1X14 | TGAAACACGGACA | GGCTAGC | CCAAAGTAGT |
| | | | TACAACGA | |
| 4 | dpp-X1-l1X13 | GAAACACGGACA | GGCTAGC | CCAAAGTAGT |
| | | | TACAACGA | |
| 5 | dpp-X1-l1X12 | AAACACGGACA | GGCTAGC | CCAAAGTAGT |
| | | | TACAACGA | |
| 6 | dpp-X1-l1Xl1 | AACACGGACA | GGCTAGC | CCAAAGTAGT |
| | | | TACAACGA | |
| 7 | dpp-X1-l1Xs1 | ACACGGACA | GGCTAGC | CCAAAGTAGT |
| | | | TACAACGA | |
| 8 | dpp-X1-l1Xs1 | CACGGACA | GGCTAGC | CCAAAGTAGT |
| | | | TACAACGA | |
| 9 | dpp-X1-l1Xs2 | ACGGACA | GGCTAGC | CCAAAGTAGT |
| | | | TACAACGA | |
| 10 | dpp-X1-l1Xs3 | CGGACA | GGCTAGC | CCAAAGTAGT |
| | | | TACAACGA | |
| 11 | dpp-X1-l1Xs4 | GGACA | GGCTAGC | CCAAAGTAGT |
| | | | TACAACGA | |
| 12 | dpp-X1-l1Xs5 | GACA | GGCTAGC | CCAAAGTAGT |
| | | | TACAACGA | |
| 13 | dpp-X1-l1Xs6 | ACA | GGCTAGC | CCAAAGTAGT |
| | | | TACAACGA | |
| 14 | dpp-X1-l1Xs7 | CA | GGCTAGC | CCAAAGTAGT |
| | | | TACAACGA | |

The following length variants of the substrate binding domain II were carried out, with a constant 12 nucleotides in the substrate binding domain I, and were tested for their cleavage properties in the cleavage assay, FIG. 3 B:

DNAzyme

| | | Substrate | | Substrate |
|---|---|---|---|---|
| Seq | | binding | Catalytic | binding |
| ID | Name | domain I | domain II | domain |
| 4 | dpp-X1-l1X13 | GAAACACGGACA | GGCTAGCTA | CCAAAGTAGT |
| | | | CAACGA | |
| 15 | dpp-X1-0X13 | GAAACACGGACA | GGCTAGCTA | CCAAAGTAG |
| | | | CAACGA | |
| 16 | dpp-X1-s1X13 | GAAACACGGACA | GGCTAGCTA | CCAAAGTA |
| | | | CAACGA | |
| 17 | dpp-X1-s2X13 | GAAACACGGACA | GGCTAGCTA | CCAAAGT |
| | | | CAACGA | |
| 18 | dpp-X1-s3X13 | GAAACACGGACA | GGCTAGCTA | CCAAAG |
| | | | CAACGA | |
| 19 | dpp-X1-s4X13 | GAAACACGGACA | GGCTAGCTA | COW |
| | | | CAACGA | |
| 20 | dpp-X1-s5X13 | GAAACACGGACA | GGCTAGCTA | CCAA |
| | | | CAACGA | |
| 21 | dpp-X1-s6X13 | GAAACACGGACA | GGCTAGCTA | CCA |
| | | | CAACGA | |
| 22 | dpp-X1-s7X13 | GAAACACGGACA | GGCTAGCTA | CC |
| | | | CAACGA | |

The minimum sequence for dpp-X1 is at least 8 nucleotides for the substrate binding domain I and at least 8 nucleotides for the substrate binding domain II. In particular with substrate binding domain I CACGGACA and substrate binding domain II CCAAAGTA.

The length of the substrate binding domains I or II can also be longer than the specified nucleotides. In one embodiment the substrate binding domains I and II are of equal length. In a further embodiment the substrate binding domains I and II are of different length.

### b) DNAzyme Dpp-X2 Shows a Good RNA Cleavage Activity of 97.76% of the Available Picornaviruses, e.g. the Rhinovirus Types HRV-1b, -16 and -29.

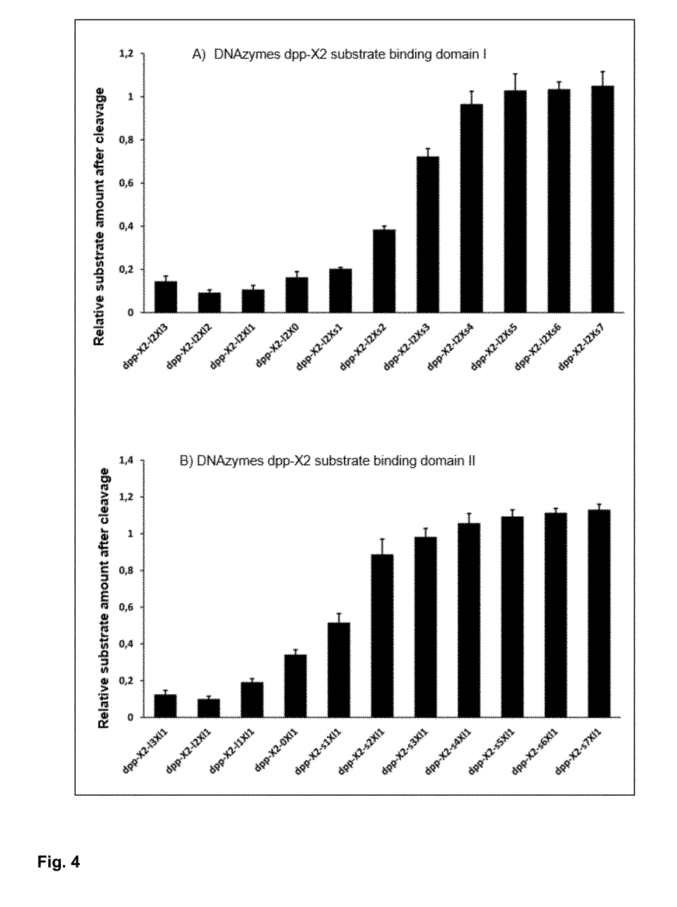

The following length variants of the substrate binding domain I were carried out, with a constant 11 nucleotides in the substrate binding domain II, and were tested for their cleavage properties in the cleavage assay, FIG. 4 A:

DNAzyme

| | | Substrate | | Substrate |
|---|---|---|---|---|
| Seq | | binding | Catalytic | binding |
| ID | Name | domain I | domain | domain II |
| 23 | dpp-X2-l2X13 | GTGAAACACGGA | GGCTAGCT | ACCCAAAGTAG |
| | | | ACAACGA | |
| 24 | dpp-X2-l2X12 | TGAAACACGGA | GGCTAGCT | ACCCAAAGTAG |
| | | | ACAACGA | |
| 25 | dpp-X2-l2Xl1 | GAAACACGGA | GGCTAGCT | ACCCAAAGTAG |
| | | | ACAACGA | |
| 26 | dpp-X2-l2X0 | AAACACGGA | GGCTAGCT | ACCCAAAGTAG |
| | | | ACAACGA | |
| 27 | dpp-X2-l2Xs1 | AACACGGA | GGCTAGCT | ACCCAAAGTAG |
| | | | ACAACGA | |
| 28 | dpp-X2-l2Xs2 | ACACGGA | GGCTAGCT | ACCCAAAGTAG |
| | | | ACAACGA | |
| 29 | dpp-X2-l2Xs3 | CACGGA | GGCTAGCT | ACCCAAAGTAG |
| | | | ACAACGA | |
| 30 | dpp-X2-l2Xs4 | ACGGA | GGCTAGCT | ACCCAAAGTAG |
| | | | ACAACGA | |
| 31 | dpp-X2-l2Xs5 | CGGA | GGCTAGCT | ACCCAAAGTAG |
| | | | ACAACGA | |
| 32 | dpp-X2-l2Xs6 | GGA | GGCTAGCT | ACCCAAAGTAG |
| | | | ACAACGA | |
| 33 | dpp-X2-l2Xs7 | GA | GGCTAGCT | ACCCAAAGTAG |
| | | | ACAACGA | |

The following length variants of the substrate binding domain II were carried out, with a 10 nucleotides in the substrate binding domain I, and were tested for their cleavage properties in the cleavage assay, FIG. 4 B:

DNAzyme

| | | Substrate | | Substrate |
|---|---|---|---|---|
| Seq | | binding | Catalytic | binding |
| ID | Name | domain I | domain | domain II |
| 34 | dpp-X2-l3Xl1 | GAAACACGGA | GGCTAGCT | ACCCAAAGTAGT |
| | | | ACAACGA | |
| 25 | dpp-X2-l2Xl1 | GAAACACGGA | GGCTAGCT | ACCCAAAGTAG |
| | | | ACAACGA | |
| 35 | dpp-X2-l1Xl1 | GAAACACGGA | GGCTAGCT | ACCCAAAGTA |
| | | | ACAACGA | |
| 36 | dpp-X2-0Xl1 | GAAACACGGA | GGCTAGCT | ACCCAAAGT |
| | | | ACAACGA | |
| 37 | dpp-X2-s1Xl1 | GAAACACGGA | GGCTAGCT | ACCCAAAG |
| | | | ACAACGA | |
| 38 | dpp-X2-s2Xl1 | GAAACACGGA | GGCTAGCT | ACCCAAA |
| | | | ACAACGA | |
| 39 | dpp-X2-s3Xl1 | GAAACACGGA | GGCTAGCT | ACCCAA |
| | | | ACAACGA | |
| 40 | dpp-X2-s4Xl1 | GAAACACGGA | GGCTAGCT | ACCCA |
| | | | ACAACGA | |
| 41 | dpp-X2-s5Xl1 | GAAACACGGA | GGCTAGCT | ACCC |
| | | | ACAACGA | |
| 42 | dpp-X2-s6Xl1 | GAAACACGGA | GGCTAGCT | ACC |
| | | | ACAACGA | |
| 43 | dpp-X2-s7Xl1 | GAAACACGGA | GGCTAGCT | AC |
| | | | ACAACGA | |

The minimum sequence for dpp-X2 is at least 6 nucleotides for the substrate binding domain I and at least 7 nucleotides for the substrate binding domain II. In particular in the substrate binding domain I CACGGA and substrate binding domain II ACCCAAA. The length of the substrate binding domains I or II can also be longer than the specified nucleotides. In one embodiment the substrate binding domains I and II are of the same length. In a further embodiment the substrate binding domains I and II are of different lengths.

### c) DNAzyme Dpp-j-9 Shows a RNA Cleavage Activity of 97.76% of the Available Picornaviruses, e.g. the Rhinovirus Types HRV-1b, -16 and -29.

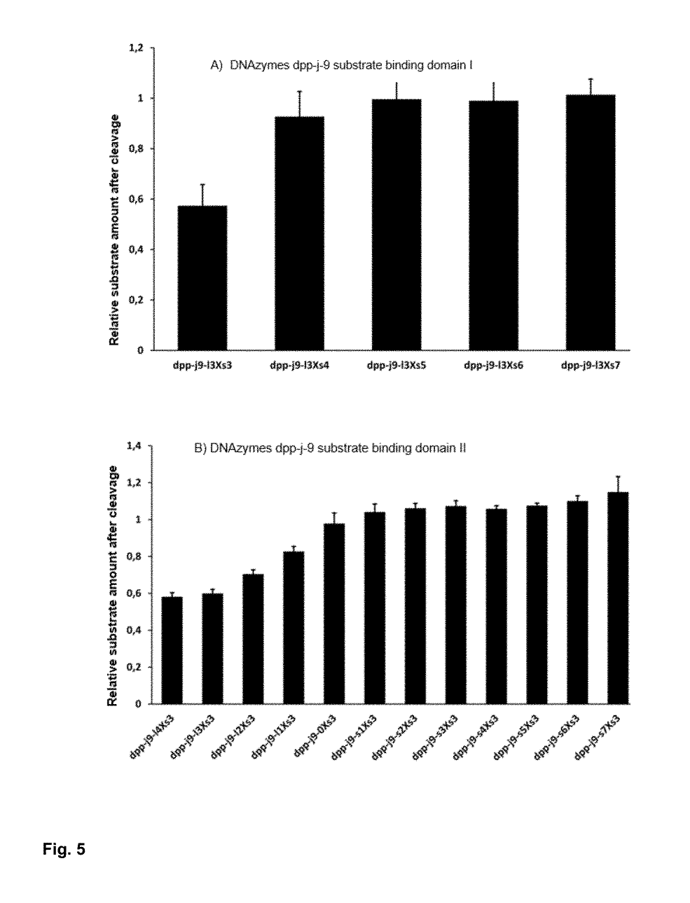

The following length variants of the substrate binding domain I were carried out, with a constant 12 nucleotides in the substrate binding domain II, and were tested for their cleavage properties in the cleavage assay, FIG. 5A:

DNAzyme

| | | Substrate | | Substrate |
|---|---|---|---|---|
| Seq | | binding | Catalytic | binding |
| ID | Name | domain I | domain | domain II |
| 44 | dpp-j-9-l3Xs3 | GAAACA | GGCTAGCTA | GGACACCCAAAG |
| | | | CAACGA | |
| 46 | dpp-j-9-l3Xs5 | AACA | GGCTAGCTA | GGACACCCAAAG |
| | | | CAACGA | |
| 47 | dpp-j-9-l3Xs6 | ACA | GGCTAGCTA | GGACACCCAAAG |
| | | | CAACGA | |
| 48 | dpp-j-9-l3Xs7 | CA | GGCTAGCTA | GGACACCCAAAG |
| | | | CAACGA | |

The following length variants of the substrate binding domain II were carried out, with a constant 6 nucleotides in the substrate binding domain II, and were tested for their cleavage properties in the cleavage assay, FIG. 5B:

DNAzyme

| | | Substrate | | Substrate |
|---|---|---|---|---|
| Seq | | binding | Catalytic | binding |
| ID | Name | domain I | domain | domain II |
| 49 | dpp-j-9-l4Xs3 | GAAACA | GGCTAGCT | GGACACCCAAAGT |
| | | | ACAACGA | |
| 44 | dpp-j-9-l3Xs3 | GAAACA | GGCTAGCT | GGACACCCAAAG |
| | | | ACAACGA | |
| 50 | dpp-j-9-l2Xs3 | GAAACA | GGCTAGCT | GGACACCCAAA |
| | | | ACAACGA | |
| 51 | dpp-j-9-l1Xs3 | GAAACA | GGCTAGCT | GGACACCCAA |
| | | | ACAACGA | |
| 52 | dpp-j-9-0Xs3 | GAAACA | GGCTAGCT | GGACACCCA |
| | | | ACAACGA | |
| 53 | dpp-j-9-s1Xs3 | GAAACA | GGCTAGCT | GGACACCC |
| | | | ACAACGA | |
| 54 | dpp-j-9-s2Xs3 | GAAACA | GGCTAGCT | GGACACC |
| | | | ACAACGA | |
| 55 | dpp-j-9-s3Xs3 | GAAACA | GGCTAGCT | GGACAC |
| | | | ACAACGA | |
| 56 | dpp-j-9-s4Xs3 | GAAACA | GGCTAGCT | GGACA |
| | | | ACAACGA | |
| 57 | dpp-j-9-s5Xs3 | GAAACA | GGCTAGCT | GGAC |
| | | | ACAACGA | |
| 58 | dpp-j-9-s6Xs3 | GAAACA | GGCTAGCT | GGA |
| | | | ACAACGA | |
| 59 | dpp-j-9-s7Xs3 | GAAACA | GGCTAGCT | GG |
| | | | ACAACGA | |

The minimum sequence for dpp-j-9 is at least 6 nucleotides for the substrate binding domain I and at least 10 nucleotides for the substrate binding domain II.

In particular in the substrate domain I GAAACA and the substrate binding domain II GGACACCCAA. The length of the substrate binding domains I or II can also be longer than the specified nucleotides. In one embodiment the substrate binding domains I and II are of the same length. In a further embodiment the substrate binding domains I and II are of different lengths.

### LEGENDS TO THE DIAGRAMS AND LIST OF REFERENCE NUMERALS

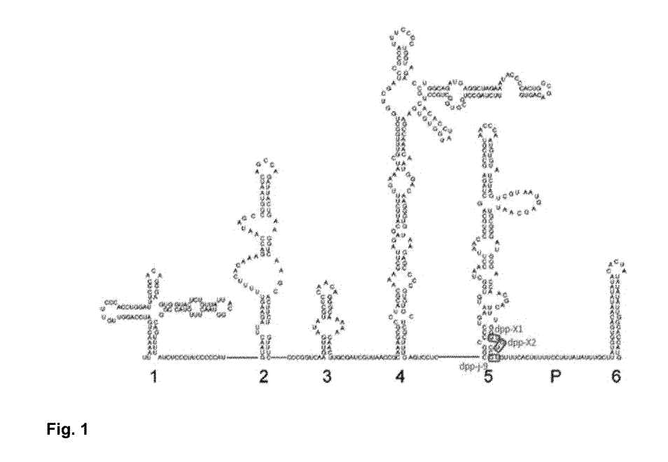

FIG. 1 shows the various serotypes that are allocated to the group of human rhinoviruses according to the current classification

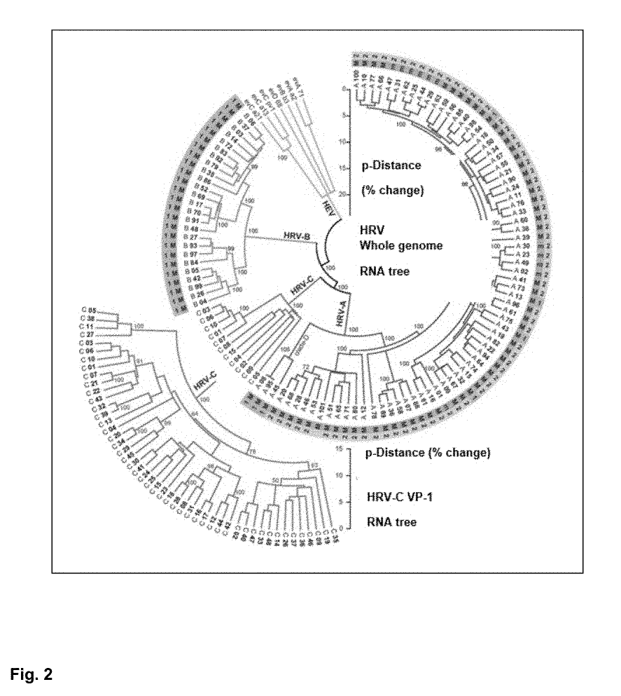

FIG. 2 shows the target sequence (Seq. ID 1) CTAGTTTGGGTGTCCGTTTC within the 5′-UTR region. This region has a very large sequence homology and low mutation rate within all available virus genomes of the picornaviruses and rhinoviruses.

The secondary structure is illustrated by the example of the human rhinovirus 2 virus with six stem-loop structures (subdomains 1-6) and the polypyrimidine tract (P) between subdomains 5 and 6. The cleavage regions of the DNAzymes dpp-X1, dpp-X2, dpp-j-9 according to the invention are enclosed by small boxes

FIG. 3 shows the cleavage activity of the length variants of the DNAzyme dpp-X1 (Seq. ID 2-22) in the cleavage assay
A) Variations of the substrate binding domain I
B) Variations of the substrate binding domain II

FIG. 4 shows the results of the length variants of the DNAzyme dpp-X2 (Seq. ID 23-43) in the cleavage assay
A) Variations of the substrate binding domain I
B) Variations of the substrate binding domain II

FIG. 5 shows the results of the length variants of the DNAzyme dpp-j-9 (Seq. ID 44-59) in the cleavage assay
A) Variations of the substrate binding domain I
B) Variations of the substrate binding domain II

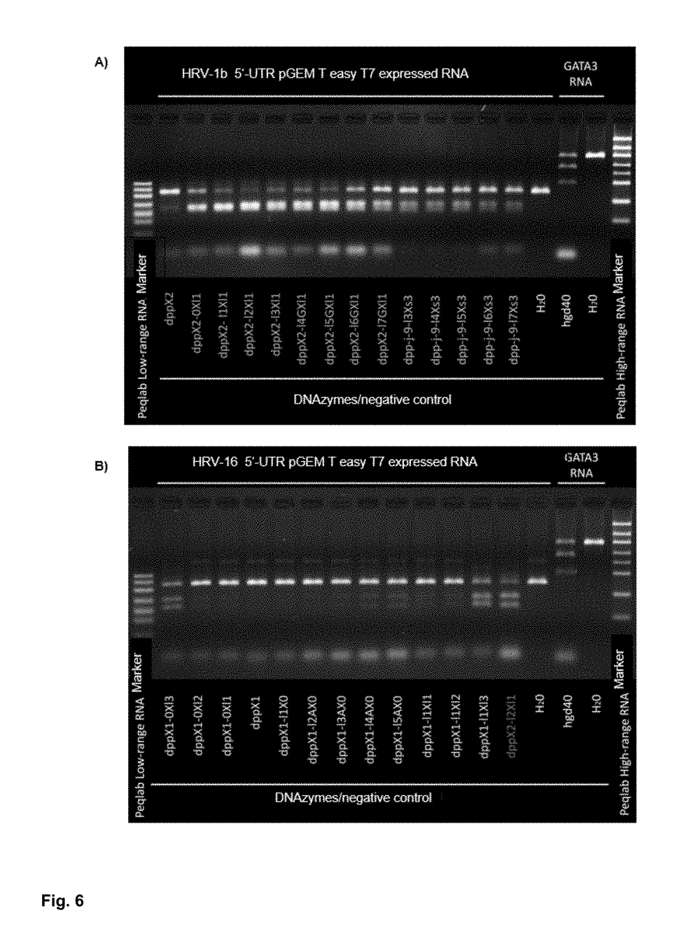

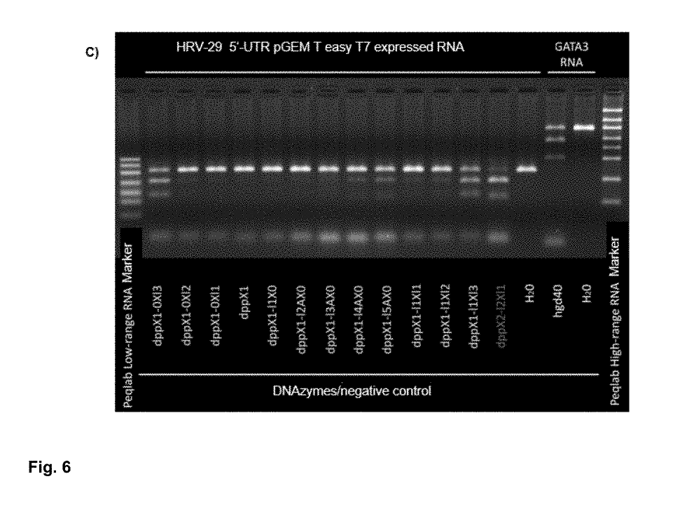

FIG. 6 shows in

A) the results of the cleavage experiments of the DNAzymes according to the invention on the HRV Serotype 1b
B) the results of the cleavage experiments of the DNAzymes according to the invention on the HRV Serotype 16
C) the results of the cleavage experiments of the DNAzymes according to the invention on the HRV Serotype 29

1. An agent for the prophylaxis and treatment of a viral infection caused by a picornavirus, the agent comprising at least one DNAzyme of the type 10-23 selected from SEQ ID NOS: 2-62, wherein the DNAzyme comprises at least one catalytic domain, a substrate binding domain I, and a substrate binding domain II, the substrate binding domains I and II being at least 80% complementary to a target sequence of an mRNA of the picornavirus.

2. The agent according to claim 1, wherein the target sequence is located in a 5′-UTR region of the picornavirus.

3. The agent according to claim 1, wherein the target sequence is the 5′-UTR region of the picornavirus according to SEQ ID NO. 1.

4. The agent according to claim 1, wherein at least one nucleotide of the substrate binding domain I or II has a modification.

5. The agent according to claim 1, wherein at least one nucleotide of the catalytic domain has a modification.

6. The agent according to claim 1, wherein the substrate binding domain I or II is at least 95% complementary to the target sequence of the mRNA of the picornavirus.

7. The agent according to claim 1, wherein the substrate binding domains I and II are different lengths.

8. The agent according to claim 1, wherein the substrate binding domains I and II are the same length.

9. The agent according to claim 1, wherein a minimum sequence of the DNAzyme according to SEQ ID NO: 2-22 or 60 is at least 8 nucleotides for the substrate binding domain I and at least 8 nucleotides for the substrate binding domain II.

10. The agent according to claim 1, wherein a minimum sequence of the DNAzyme according to SEQ ID NO: 23-43 or 61 is at least 6 nucleotides for the substrate binding domain I and at least 7 nucleotides for the substrate binding domain II.

11. The agent according to claim 1, wherein a minimum sequence of the DNAzyme according to SEQ ID NO: 44-59 or 62 is at least 6 nucleotides for the substrate binding domain I and at least 10 nucleotides for the substrate binding domain II.

12. The agent according to claim 1, further comprising at least one anti-inflammatory agent, immune modulator, anti-asthmatic agent, analgesic agent, antipyretic agent, bronchodilator, or a combination thereof.

13. The agent according to claim 1, further comprising at least one antiparasitic active ingredient, antibacterial active ingredient, antimycotic active ingredient, antiviral active ingredient, or a combination thereof.

14. A method of preventing rhinitis, a head cold, asthma, chronic obstructive pulmonary disease (COPD), or a viral infection of the upper respiratory tract in a subject, the method comprising administering a prophylactically effective amount of the agent of claim 1 to the subject.

15. A method of treating rhinitis, a head cold, asthma, chronic obstructive pulmonary disease (COPD), or a and viral infection of the upper respiratory tract in a subject, the method comprising administering a therapeutically effective amount of the agent of claim 1 to the subject.

16. The agent according to claim 1 wherein the agent is formulated for administration by inhalation, as a spray, as drops, orally or in tablet form.

17. The method according to claim 14, wherein the agent is administered by inhalation, as a spray, as drops, orally, or in tablet form.

18. The method according to claim 14, wherein the agent is administered in combination with at least one anti-inflammatory agent, immune modulator, anti-asthmatic agent, analgesic agent, antipyretic agent, bronchodilator, or a combination thereof.

19. The method according to claim 15, wherein the agent is administered by inhalation, as a spray, as drops, orally, or in tablet form.

20. The method according to claim 15, wherein the agent is administered in combination with at least one anti-inflammatory agent, immune modulator, anti-asthmatic agent, analgesic agent, antipyretic agent, bronchodilator, or a combination thereof.
